(19) United States
(12) Patent Application Publication
(10) Pub. No.: US 2021/0361661 A1
(43) Pub. Date: Nov. 25, 2021

(54) SEMI-SOLID CAFFEINATED COMPOSITION AND METHODS OF MAKING AND USING THEREOF

(71) Applicant: SEATTLE GUMMY COMPANY, Seattle, WA (US)

(72) Inventors: Feng WAN, Issaquah, WA (US); William CARLSON, Shoreline, WA (US); Bradley FITCH, Seattle, WA (US)

(21) Appl. No.: 16/963,230

(22) PCT Filed: Jan. 14, 2019
(86) PCT No.: PCT/US19/13540

§ 371 Date: Jul. 19, 2020

(51) Int. Cl. A61K 31/522; A61K 36/82; A61K 36/77; A61K 36/185; A61K 36/74; A61K 9/00; A61K 47/69; A61K 47/46; A61K 47/36; A61K 47/26; A61K 31/355; A61K 31/145; A61K 31/198; A61K 31/12; A61K 31/353; A61K 36/258; A61K 36/16; A61K 36/8962; A61K 31/122; A61K 31/047; A61K 31/07; A61K 31/375; A61K 33/04; A61K 33/06; A61K 33/26; A61K 33/30; A61K 33/14; A61K 33/34; A61K 33/24; A61K 33/32; A61K 33/18; A61K 33/42; A61K 31/593; A61K 36/9066; A61K 36/9068; A61K 36/87; A61K 36/45; A61K 36/537; A61K 36/481; A61K 36/482; A61K 36/488; A61K 36/88; A61K 36/46; A61K 36/536

(52) U.S. Cl. CPC A61K 31/522; A61K 36/536; A61K 36/77; A61K 36/185; A61K 36/74; A61K 9/0056; A61K 47/6951; A61K 47/46; A61K 47/36; A61K 47/26; A61K 31/355; A61K 31/145; A61K 31/198; A61K 31/12; A61K 31/353; A61K 36/258; A61K 36/16; A61K 36/8962; A61K 31/122; A61K 31/047; A61K 31/07; A61K 31/375; A61K 33/04; A61K 33/06; A61K 33/26; A61K 33/30; A61K 33/14; A61K 33/34; A61K 33/24; A61K 33/32; A61K 33/18; A61K 33/42; A61K 31/593; A61K 36/9066; A61K 36/9068; A61K 36/87; A61K 36/45; A61K 36/537; A61K 36/481; A61K 36/482; A61K 36/488; A61K 36/88; A61K 36/46; A61K 36/82

(57) ABSTRACT

A semi-solid composition comprising a gelling component in a sufficient amount to provide a cohesive gelled product, a caffeinated component comprising caffeine, wherein the caffeinated component is bitter, and a complexing component, wherein the complexing component is configured to reduce the bitterness of the caffeinated component by complexing with the caffeinated component.

### CROSS REFERENCE TO RELATED APPLICATIONS

This application claims the benefit of the filing date of U.S. Provisional Application Ser. No. 62/617,301, filed Jan. 15, 2018, U.S. Provisional Application Ser. No. 62/683,523, filed Jun. 11, 2018, U.S. Provisional Application Ser. No. 62/689,106, filed Jun. 23, 2018, and the entire disclosures of which are incorporated by reference herein.

### TECHNICAL FIELD

This application relates to semi-solid edible or chewable compositions with one or more bioactive incorporated therein.

### BACKGROUND

Unless otherwise indicated herein, the materials described in this section are not prior art to the claims in this application and are not admitted being prior art by inclusion in this section.

Caffeine is a bitter-tasting compound. The xanthine core of caffeine contains two fused rings, a pyrimidinedione and imidazole. Pharmacologically, caffeine is a central nervous system stimulant. The compound is known to cross the blood brain barrier and reversibly blocks the action of adenosine on its receptor and consequently prevents the onset of drowsiness induced by adenosine. Caffeine also stimulates certain portion of the autonomic nervous system. The undesired effects from caffeine ingestion are common, including mild anxiety, jitteriness, hear palpitation, increased blood pressure, insomnia, increase sleep latency and reduced coordination. Researches have positively associated caffeine use with anxiety and panic disorders

Gummy are a category of semi-solid chewable confectionary product made from gelling agent such as gelatin. There are numerous attempts incorporating caffeine into gummy products. However, because of the bitter tastes from caffeine, the amounts of caffeine that can be incorporate into gummies have been very limited so far.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides semi-solid composition. The semi-solid composition may be chewable. In one embodiment, the semi-solid composition may be a gummy composition. In one embodiment, the semi-solid chewable composition may be gum composition.

In one embodiment, the semi-solid composition includes a gelling component in a sufficient amount to provide a cohesive gelled product, a caffeinated component comprising caffeine, and a complexing component. The caffeinated component may be bitter. The complexing component may be configured to reduce the bitterness of the caffeinated component by complexing with the caffeinated component.

In one embodiment, the caffeinated component comprises caffeine, green coffee bean powder or extract, green tea powder or extract, white tea powder or extract, black tea powder or extract, guarana powder or extract, yerba mate powder or extract, cola nut powder or extract, cacao powder or extract, coffee powder or extract, or a combination thereof. In one embodiment, the caffeinated component comprises a plant extract or powder containing at least 3%, 4%, 5%, 10%, 20%, 30%, 40%, or 50% of caffeine. In one embodiment, the caffeinated component comprises cacao derivatives, comprising theobromine, polyphenol, flavonoids, or a combination thereof.

In one embodiment, the complexing component may be configured to complexing with the caffeinated component to form a complex therefore reducing the bitterness of the caffeinated component. The complexing may be through coordinating, chelating, complexing, hydrogen-bonding, dipole-dipole interaction, van-der waals interaction, electrostatic interaction, or a combination thereof.

In one embodiment, the complexing component comprises nucleic acid, nucleic acid bases, nucleotide, fruit power, protein, peptide, cluster dextrin, cyclodextrin, polydextrose, polyethylene glycol, fatty acids, waxes, zeolite, chitosan, poly N-acetylglucosamine, or a combination thereof. In one embodiment, the complexing component is configured to complex with caffeine. In one embodiment, the complexing component is configured to complex with theobromine, polyphenol, flavonoids, or a combination thereof.

In one embodiment, the semi-solid composition comprises at least 0.5% of the complexing component by weight. In one embodiment, the semi-solid composition comprises at least 12% of the complexing component by weight. In one embodiment, the complexing component comprises cyclodextrin and the semi-solid composition comprises at least 0.2%, 0.3%, 0.5%, 0.8%, 1%, 1.5%, or 2% of cyclodextrin. In one embodiment, the complexing component comprises strawberry powder and wherein the semi-solid composition comprises at least 0.2%, 0.25%, 0.5%, 0.8%, 1% or 2% of strawberry powder.

In one embodiment, the gelling composition comprises gelatin, starch, pectin, gellan gum, gum Arabic, carrageenans, guar, agar, alginate, locust bean gum, xanthan, or a combination or derivatives thereof. In one embodiment, the gelling composition comprises pectin and gelatin in a ratio from about 10:1 to about 1:1. In one embodiment, the gelling composition consists essentially of pectin. In one embodiment, the gelling composition comprises gelatin and starch in a ratio from about 100:1 to about 1:100. In one embodiment, the gelling composition comprises essentially of gelatin.

The semi-solid composition may include at least 0.5% caffeine by weight. In one embodiment, the semi-solid composition comprises at least 1% caffeine by weight. In one embodiment, the semi-solid composition comprises at least 1.5%, 2%, 2.5% or 3% caffeine by weight.

The semi-solid composition may further include a modulating composition, an antioxidant composition, a vitamin composition, a mineral composition, an amino acid composition, an herb composition, a prebiotic composition, a probiotic composition, or a combination thereof.

The modulating composition is configured to reduce or counter act caffeine's side effect including without limitation jittery, anxiety, heart palpitation, raised blood pressure, or a combination thereof. In one embodiment, the modulating composition comprises adrenergic receptor antagonist, adrenergic receptor agonist, calcium channel blocker, ACE inhibitor, angiotensin II receptor antagonist, aldosterone antagonist, vasodilator, centrally acting adrenergic compound, PAF receptor inhibitor, or a combination thereof. In one embodiment, the modulating composition comprises compounds that are configured to antagonize platelet activation factor (PAF), improve alpha-2 adrenoreceptor activity, catechol-O-methyl transferase (COMT), dilate blood vessels, increase level of 5-hydroxytryptamine (5-HT) in the hippocampus, or a combination thereof.

In some embodiments, the modulating composition comprises gingko biloba, cocoa, theobromine, theanine, piraletam, citicoline, blubbery extract or isolates, arginine, vitamin E, bacopa, curcumin, ginseng, citrulline, icariin, forsklin, S-denosyl-L-methionine, quercetine, taurine, grape seed extract, or isolates, extracts or derivatives thereof.

In some embodiments, the modulating composition comprises danshen, Angelica sinesis (danggui), safflower, red clover, wild yam, American ginseng, valerian, St. John's wort, goldenseal, turmeric, grape seed, slippery elm, cayenne, Devil's Claw, feverfew, Jamaica dogwood, linden, willow bark, peppermint, barberry, celery seed, dandelion, Gotu Kola, bilberry, Asian ginseng, green tea, rosemary, Siberian ginseng, saw palmetto, ashwagandha, bacopa monnieri, hordenine, isoflavones, kava kava, cat's claw (Uncaria tomentosa, Uncaria guianensis), lavender, cinnamon (Cinnamomum verum), Yarrow (Achilea millefolium), hawthorn, garlic, Buchu (Agathosma betulina), prickly custard apple (Annona muricata), Cassia occidentalis (Coffee weed), Hibiscus sabdariffa (Roselle), blackberry, blueberry, raspberry, acai berry, goji berry, or plant parts, extract, isolates or powders thereof.

In one embodiment, the modulating composition comprises magnesium, L-theanine, theothromine, piraletam, citicoline, flavonoids, quinones, blubbery extract or isolates, arginine, vitamin E, bacopa, curcumin, ginseng, citrulline, icariin, forsklin, S-denosyl-L-methionine, quercetine, taurine, or isolates, extracts or derivatives thereof.

In some embodiments, the modulating composition comprises Ginkgo biloba leave extract, Ginkgo biloba flavonoids, cocoa flavonoids, or a combination thereof. In some embodiments, the modulating composition comprises epicatechin, catechin, quercetin, kaempferol, isorhamnetin, amentoflavone, bilobetin, isoginkgetin, ginkgetin, sciadopitysin, or a combination thereof.

In some embodiments, the modulating composition comprises gingko biloba, its isolates, extracts or powders thereof. In some embodiments, the modulating composition comprises danshen isolates, extracts or powders thereof. In some embodiments, the modulating composition comprises Angelica sinesis (danggui) isolates, extracts or powders thereof. In some embodiments, the modulating composition comprises grade seed isolates, extracts or powders thereof.

In one embodiment, the antioxidant composition comprises vitamin A, vitamin E, vitamin C, beta-carotene, alpha-carotene, lycopene, lutein, folic acid, gallic acid, resveratrol, quinone, Coenzyme Q10, selenium, selenium yeast, phenolics, polyphenols, anthocyanins, flavonoids, astaxanthin, canthaxanthin, cryptoxanthin, anthracenes, carotenoids, zeaxanthin, curcumin, or derivatives thereof.

In one embodiment, the vitamin composition comprises vitamin A, B, C, D, E, K or a combination thereof.

In one embodiment, the mineral composition comprises salts of calcium, iron, zinc, magnesium, sodium, chloride, potassium, copper, molybdenum, manganese, phosphorus, iodine, nickel, or selenium, or a combination thereof.

In one embodiment, the amino acid composition comprises an essential amino acid, a branch-chain amino acid, a stimulant amino acid, or its derivative thereof.

In one embodiment, the herbal composition comprises ginko biloba, turmeric, ginger, astragalus, Prunella vulgaris, Pueraria montana var. lobata, Salvia miltiorrhiza, Coptis chinensis, Eucommia ulmoides Oliver, cranberry, blackberry, elderberry extract, cranberry, blueberry, grapeseed, saffron, Sangre de grado (dragon's blood), its extract, powder or derivative thereof.

In one embodiment, the prebiotic composition comprises gum arabic, chicory root, wheat bran, resistant starch, mannose oligosaccharide, acacia gum, inulin, galacto-oligosaccahride, guar gum, Artichoke fiber, fructo-ligosaccharide, or a combination thereof.

The probiotic composition comprises bifidobacteria, lactic acid bacteria, or a combination thereof. In one embodiment, the probiotic composition comprises Bifidobacterium lactis, Bifidobacterium longum, Lactobacillus acidophilus, Lactobacillus paracasei, Lactobacillus plantarum, Lactobacillus rhamnosus, Bacillus coagulans, Bifidobacterium bifidum, Lactobaccillus casei, Lactobaccillus gasseri, Lactobacillus salivarius, Lactobacillus bulgarius, or a combination thereof.

The semi-solid composition may further include an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the sweetener comprises erythritol, xylitol, sucrose, fructose, glucose, maltose, juice or juice concentrate, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

In one embodiment, the flavoring agent comprises vanilla, chili oil, gingerol, peperine, capsaicin, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, rosemary oil, lavender oil, sage oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, cocoa, pineapple flavor, or mixtures or derivatives thereof.

In one embodiment, the semi-solid gummy composition may include a sugar composition. The sugar composition may function as a sweetener, as a bonding agent assisting the gelation of the gelling component, or a combination thereof. The sugar composition may include high glycemic index sugars. In one embodiment, the sugar composition comprises sugars with a glycemic index greater than 50, 60, 70, 80, 90, 100, 120, or 150. Examples high glycemic index sugar include without limitation sucrose, maltose, maltotriose, maltodextrin, dextrin, dextrose or glucose. In one embodiment, the sugar composition comprises sucrose, fructose, glucose or a combination thereof.

In one embodiment, the sugar composition may include low glycemic sugars. In one embodiment, the sugar composition comprisessugars with a glycemic index less than 50, 40, 30 or 20. Examples of low glycemic index sugar include without limitation fructose, trehalose, palatinose, psicose, tagatose, sorbose. In one embodiment, the sugar composition comprises trehalose, palatinose, psicose, tagatose, sorbose, or a combination thereof. In one embodiment, the sugar composition comprises trehalose and palatinose. In one embodiment, the sugar composition comprises palatinose and psicose. In one embodiment, the sugar composition comprises trehalose and psicose.

The semi-solid gummy composition may have a glycemic index from about 8 to about 170. In one embodiment, the semi-solid gummy composition has a glycemic index of more than 60. In one embodiment, the semi-solid gummy composition has a glycemic index of more than 90. In one embodiment, the semi-solid gummy composition has a glycemic index of not more than 30. In one embodiment, the semi-solid gummy composition has a glycemic index of not more than about 20. In one embodiment, the semi-solid gummy composition has a glycemic index of not more than about 15.

In one embodiment, the semi-solid gummy composition may be substantially free of sucrose, fructose, glucose, or a combination thereof. In one embodiment, the gummy composition may be substantially free of sugar substitutes. In one embodiment, the gummy composition may be substantially free of artificial sweeteners. In one embodiment, the gummy composition may be substantially free of sugar alcohols.

In one embodiment, the semi-solid gummy composition may be a sugar free composition. In one embodiment, the semi-solid gummy composition may include a sugar alcohol composition. In one embodiment, the sugar alcohol composition may act as a sweetener, a bonding agent for assisting the gelation of the gelling component, or both. Examples sugar alcohol may include glycerol, sorbitol, mannitol, xylitol, erythritol, isomalt, or hydrogenated starch hydrolyses. In one embodiment, the semi-solid gummy composition may be gelatin-based gummy with a sugar substitute (such as stevia) as sweetener leading to a sugar free formulation. In one embodiment, the semi-solid gummy composition comprises a sugar alcohol as the gelling component and non-caloric sweetener or sugar substitute as sweetener.

The semi-solid gummy composition may include a food acid composition. In one embodiment, the food acid composition may act to impart acidic or astringent flavor, facilitate the gelling of the gelling component, or both. In one embodiment, the food acid composition may include citric acid, malic acid, ascorbic acid, lactic acid, galactic acid, glutamic acid, tartaric acid, propionic acid, butyric acid, valeric acid, gluconic acid, isocitric acid, succinic acid, fumaric acid or a combination there. In one embodiment, the food acid composition may include citric acid, malic acid, or a combination there.

The semi-solid gummy composition may include a buffer composition. The buffer composition acts to buffer the pH of the composition when combined with one of the above acid examples, facilitate the gelling, or both. In one embodiment, the buffer composition comprises sodium citrate, potassium citrate, calcium citrate, sodium hydroxide, potassium hydroxide, calcium hydroxide, sodium carbonate, sodium bicarbonate, potassium carbonate, potassium bicarbonate, a combination thereof.

The pH of the composition may be acid, neutral or slightly basic. In one embodiment, the pH of the semi-solid composition may be from about 3 to about 5. The semi-solid gummy composition may have a pH from about 2 to about 6. In one embodiment, the pH of the composition is about 2.7 to about 2.9. In one embodiment, the pH of the composition is about 3.0 to about 3.4.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the representative nucleic acid structure;

FIG. 2 shows some example bitter tasting chemical components from plants including without limitation phenylethylamine, phenolics, anandamide, serotonin, and caffeine;

FIG. 3 shows the chemical structure of alpha-, beta- and gamma-cyclodextrin molecules; and

FIG. 4 shows the hydrophobic interior cavity of the cyclodextrin complex with a representative hydrophobic bitter tasting compound to form an inclusion complex.

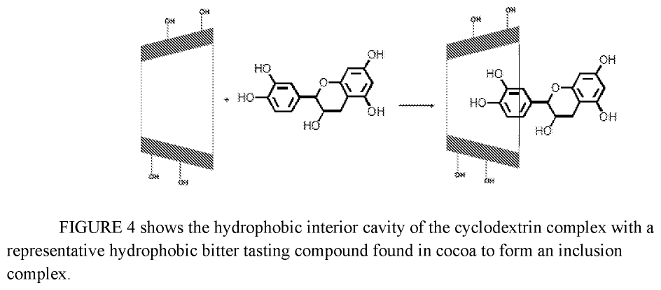

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The application provides semi-solid composition that contains a high percentage of caffeine. In one embodiment, the application provides semi-solid chewable composition that contains high percentage of caffeine without the caffeine's bitterness.

In one aspect, the application provides a semi-solid composition. In one embodiment, the semi-solid composition comprises a gelling component in a sufficient amount to provide a cohesive gelled product. In one embodiment, the semi-solid composition comprises a caffeinated component comprising caffeine. In one embodiment, the caffeinated component is bitter. In one embodiment, the semi-solid composition comprises a complexing component. In one embodiment, the complexing component is configured to reduce the bitterness of the caffeinated component by complexing with the caffeinated component.

In one embodiment, caffeine may be natural, synthetic, or a combination thereof. In one embodiment, the caffeinated component consists essentially of caffeine

In one embodiment, the caffeinated component comprises a caffeine-containing plant extract or powder. In one embodiment, the caffeine-containing plant extract or powder may contain caffeine at a concentration of at least 3%, 5%, 8%, 10%, 20%, 40%, 50%, 60%, 70%, 80%, 90%, 98%, or 99%. In one embodiment, the caffeine-containing plant extract or powder may be green coffee bean powder or extract, green tea powder or extract, white tea powder or extract, black tea powder or extract, guarana powder or extract, yerba mate powder or extract, cola nut powder or extract, cocoa powder or extract, coffee powder or extract, or a combination thereof.

In one embodiment, the caffeinated component comprises caffeine and cocoa powder. In one embodiment, the caffeinated component consists essentially of caffeine and cocoa powder or extract. In one embodiment, caffeine and cocoa powder or extract has a ratio from about 3:1 to about 1:5. In one embodiment, the caffeine and cocoa powder or extract has a ratio from about 1:4 to about 1:2. In one embodiment, the caffeinated component comprises cacao derivatives. In one embodiment, the cacao derivative may be theobromine, polyphenol, flavonoids, or a combination thereof.

In one embodiment, the caffeinated component comprises caffeine, guarana powder or extract, or a combination thereof. In one embodiment, the caffeinated component consists essentially of guarana powder or extract. In one embodiment, the guarana powder or extract contains at least 4%, 8%, 10%, 30%, 50%, 80%, 95%, 98% or 99% of caffeine.

In one embodiment, the caffeinated component comprises coffee powder or extract. In one embodiment, the coffee powder or extract contains about 3%, 4%, 8%, 10%, 30%, 50%, 80%, 95%, 98% or 99% of caffeine. In one embodiment, the caffeinated component comprises caffeine and green coffee bean powder or extract. In one embodiment, the weight ratio between caffeine and green coffee bean powder or extract is from about 1:100 to about 1:2.

In one embodiment, the total amount of caffeine content in the composition includes caffeine and the caffeine content from a plant extract at a ratio from about 10:1 to about 1:10. In one embodiment, the ratio is 1:1. In one embodiment, the ratio is 3:1. In one embodiment, the ratio is 1:3.

In one embodiment, the caffeinated component comprises green tea powder or extract. In one embodiment, the green tea powder or extract contains about 3%, 4%, 8%, or 10% of caffeine. In one embodiment, the composition comprise caffeine and the green tea powder at a weight ratio from about 1:100 to about 1:5.

The complexing component is capable of interacting with the caffeinated component through coordinating, chelating, complexing, hydrogen-bonding, dipole-dipole interaction, van-der waals interaction, electrostatic interaction, or a combination thereof. Through complexing with the caffeinated component, the complexing component acts to reduce the bitterness of the caffeinated component.

In one embodiment, the complexing component may contain polymeric molecules having a MW of at least 30 kDa. The polymeric molecule may possess tertiary structure capable of complexing with or fitting the caffeinated component into the structure through hydrogen-bonding, dipole-dipole interaction, van-der waals interaction, or a combination thereof. In one embodiment, the complexing component is configured to complex with caffeine. In one embodiment, the complexing component is configured to complex with theobromine, polyphenol, flavonoids, or a combination thereof.

In one embodiment, the complexing component contains nucleic acid, nucleotide, nucleic acid bases, fruit power, protein, peptide, cluster dextrin, cyclodextrin, polydextrose, polyethylene glycol, fatty acids, waxes, zeolite, chitosan, poly N-acetylglucosamine, or a combination thereof. In one embodiment, the complexing component comprises DNA, RNA, protein, peptide, resistant starch, porphyrin, polyunsaturated hydrocarbons, polyunsaturated fatty acids, mica, talc, zeolite, silica, cellulose, lignin, plant particles, MOF, calcium carbonate, diatomaceous earth, or a combination thereof.

Nucleic acid may be DNA, RNA, or a combination thereof. Nucleic acid may be extracted from various lifeforms or synthetic. In one embodiment, nucleic acid may have a molecule weight from about 0.2 kDa to about 1000 kDa. Examples of nucleic acids include both DNA and RNA derived from natural sources such as fruit. In one embodiment, the complexing agent comprise an adenosine rich nucleic acid. In one embodiment, the complexing agent comprises thiamin rich nucleic acid. In one embodiment, the complexing component comprises adenosine, cytosine, guanine, thiamine, uracil, or a derivative thereof.

The complexing agent may also include nucleotides or nucleic acid bases. Example nucleic acid bases may include adenine, cytosine, guanine, thymine, and uracil. In one embodiment, the complexing agent comprises adenosine, in which the caffeine molecule is capable of pairing through hydrogen bonding, similar to the adenosine and thiamin base pair formation.

Fruit powder may be strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, asci fruit powder, cashew false fruit powder, monk fruit powder, dragon fruit powder, passion fruit powder, coconut powder, guava powder, cranberry powder or blueberry powder. In one embodiment, the semi-solid composition comprises at least 0.035%, 0.05%, or 0.1%, 0.2%, 0.3% of strawberry powder. In one embodiment, the semi-solid composition comprises at least 0.05%, 0.1%, 0.2%, or 0.3% of comprises orange peel or pulp powder. In one embodiment, the semi-solid composition comprises at least 0.05%, 0.1%, 0.2%, or 0.3% of lemon peel or pulp powder. In one embodiment, the semi-solid composition comprises at least 0.065%, 0.1%, 0.1%, 0.3%, 0.4%, 0.5%, 0.75%, 1%, or 2% of goji berry powder.

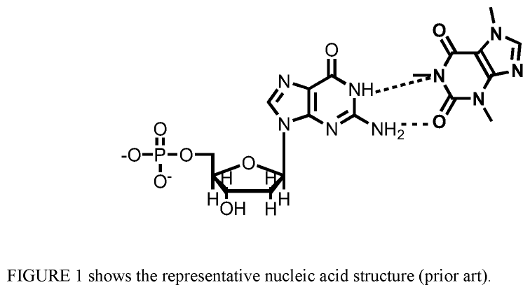

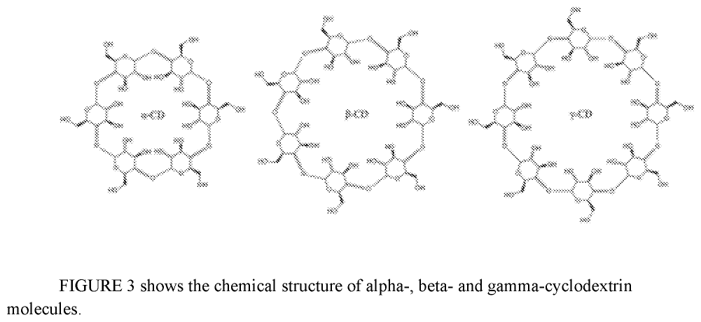

In one embodiment, the complexing component comprises a nucleic acid molecule. In one embodiment, the nucleic acid molecule may be a DNA molecule (FIG. 3). The DNA molecule may form a DNA-caffeine complex therefore reducing or modulating the bitterness of caffeine. In one embodiment, the DNA-caffeine complex may have an arrangement in which the caffeine molecule is complexed with DNA double helix with an orientation parallel to the bases. In one embodiment, the caffeine molecule complexes with DNA double helix through hydrogen-bonding. In one embodiment, the complexing component comprises DNA molecules from plant source. In one embodiment, the complexing component comprises strawberry DNA having the structure as shown in FIG. 1.

Caffeine is similar in structure to DNA and RNA base pairs. Being similar in structure and functionality, the caffeine molecule is able to hydrogen bond with the base pairs and form a DNA-caffeine complex. The complex helps to lessen the bitterness of the caffeine.

Protein or peptide may have a MW from about 0.5 kda to about 1000 kda. In one embodiment, the peptide may be polylysine. In embodiment, the peptide may have MW of not more than 30 kDa. In one embodiment, the peptide may include FVDVT, AGPHGPPGKDGR, D4E1, GLP-1, collagen, or a combination thereof.

Cluster dextrin may have a MW from about 1 kDa to about 400 kDa. In one embodiment, the complexing component comprises cluster dextrin and the semi-solid composition comprises at least 0.1% of cluster dextrin.

Cyclodextrin may be alpha, beta, or gamma. In one embodiment, cyclodextrin may have a MW from about 950 g mol-1 to about 3400 g mol-1. In one embodiment, the semi-solid composition comprises at least 0.1% of cyclodextrin. In one embodiment, cyclodextrin comprises alpha, beta, gamma-cyclodextrin or a combination thereof. In one embodiment, the complexing component comprises alpha-cyclodextrin, beta-cyclodextrin, gamma-cyclodextrin, or a combination thereof. In one embodiment, the complexing component consists essentially of alpha-cyclodextrin. In one embodiment, the complexing component consists essentially of beta-cyclodextrin. In one embodiment, the complexing component consists essentially of gamma-cyclodextrin. In one embodiment, the semi-solid composition comprises from about 0.05% to about 1% of gamma-cyclodextrin. In one embodiment, the semi-solid composition comprises at least 0.05%, 0.1%, 0.2% or 0.5% of gamma-cyclodextrin.

Cyclodextrins (sometimes called cycloamyloses) are a family of compounds made up of sugar molecules bound together in a ring (cyclic oligosaccharides). Cyclodextrins are composed of 5 or more α-D-glucopyranoside units linked 1->4. Typical cyclodextrins contain a number of glucose monomers ranging from six to eight units in a ring, creating a cone shape. The largest cyclodextrin contains 32 units of1,4-anhydroglucopyranoside.

Cyclodextrin molecules may have substitution on the ring of α-D-glucopyranoside units. Some examples of moiety substitution on the ring of α-D-glucopyranoside units include hydroxypropyl, methyl, ethyl, acetyl, butyrate, iodo, amino, azido, carboxymethyl and the like. Substitution upon the α-D-glucopyranoside units can modify the interior cavity of the cyclodextrin.

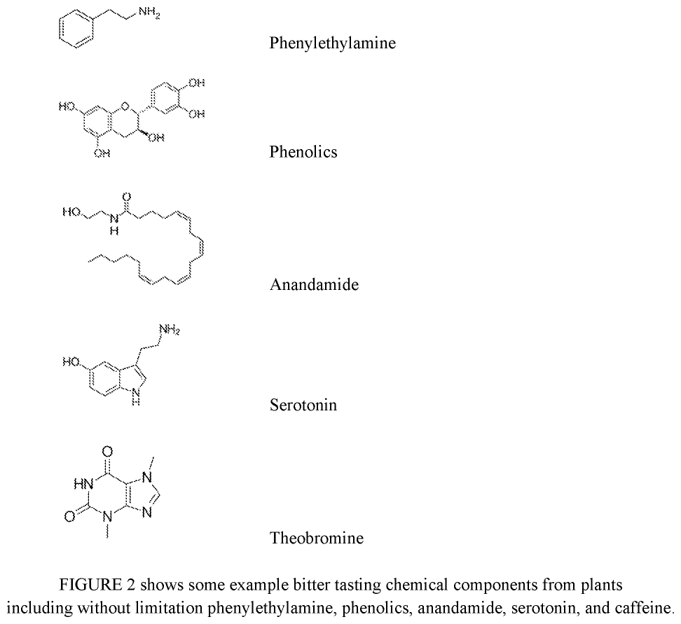

There are many chemical components in plants. These chemicals can affect the overall odor, taste, and sensation of the plant upon ingestion. Many of these chemical components contribute to the bitterness taste. FIG. 2 shows some example bitter tasting chemical components from plants including without limitation phenylethylamine, phenolics, anandamide, serotonin, and theobromine. Complexing these bitter tasting compounds help reduces the amount of bitter causing chemicals in the semi-solid gummy mixture and allows for the inclusion of more actives such as caffeine.

α (alpha)-cyclodextrin is a 6-membered sugar ring molecule. β(beta)-cyclodextrin is a 7-membered sugar ring molecule. γ (gamma)-cyclodextrin is a 8-membered sugar ring molecule. Alpha-, beta-, and gamma-cyclodextrin are all generally recognized as safe by the FDA. Alpha is limited to 3% by weight of the product being consumed whereas beta has a dietary limit of 50 mg/kg. There are no dietary limits on gamma-cyclodextrin. The interior of the cyclodextrin, be it alpha, beta or gamma, is extraordinarily hydrophobic while the exterior of the cyclodextrin is hydrophilic. FIG. 3 shows the structure of various cyclodextrin molecules.

In one embodiment, the complexing component comprises cyclodextrin. The cyclodextrin molecules may form inclusion complex with hydrophobic bitter tasting molecules. In one embodiment, the bitter tasting compounds are complexed inside the cone shape of the cyclodextrin molecule, therefore shields bitter taste. FIG. 2 shows the interior cavity of the cyclodextrin, which is hydrophobic. The hydrophobic interior cavity allows the cyclodextrin to capture bitter tasting compounds that are hydrophobic. The hydrophobic cavity of the cyclodextrin allows for formation of inclusion complexes between a bittering agent and cyclodextrin which removes the bitter taste imparted by the bittering agent. The inclusion complexes allow for more actives, such as caffeine, to be added without impacting overall bitterness levels of the semi-solid composition.

In one embodiment, cyclodextrins may enhance caffeine permeability through mucosal tissues, allowing the quick absorption and action through the chewing of the semi-solid gummy composition.

In one embodiment, the complexing component may comprise cyclodextrin and a biophenol. The biophenol may form complex with cyclodextrin and caffeine. In one embodiment, the biphenol-caffein-cyclodextrin complex may be more stable than caffeine-cyclodextrin complex. In one embodiment, the biophenol comprises tyrosol, oleuropein, or a combination thereof. In one embodiment, the biophenol comprises polyphenols such as flavonoids.

Fatty acids may be saturated or unsaturated. Example fatty acids include without limitation coconut oil or fat, palm oil or fat, cocoa butter, shea butter, lard, bacon fat, milk fat, linseed oil, flax seed oil, hemp oil, safflower oil, cotton seed oil, avocado oil, grape seed oil, olive oil and the like.

Waxes may be carnauba wax, bee's wax, paraffin wax, rice bran wax, sugar cane wax, shellac, or resin or any combination.

The complexing component may include plant particles or powder. In one embodiment, the plant particles are derived from husk, seed, seed shell, nut, nut shell, fruit, flower, stem, leaf, rice husk, nut shell, woody root, stem or leaves, corn husk, oat husk, grain husk, yeast, mushroom, berry seed, raspberry seed, blackberry seed, blueberry seed, strawberry fruit, chili, pepper, or a combination thereof. In one embodiment, the plant particles comprised defatted berry seed particles. In one embodiment, the plant particles have a particle size from about at least 70 mesh. In one embodiment, the plant particle has a particle size from about 70 to about 200 mesh. In one embodiment, the plant particle has a particle size of not greater than about 200 mesh.

The semi-solid composition may include at least 0.01% of the complexing component by weight. In one embodiment, the semi-solid composition may include from about 0.5% to about 10.0% the complexing component by weight. In one embodiment, the semi-solid composition may include from about 1% to about 12% of the complexing component by weight.

In one embodiment, the molar ration of complexing component and the caffeinated component may be from about 1:1 to about 100:1. In one embodiment, the molar ration of complexing component and the caffeinate component is at least 1:1, 2:1, 5:1, 10:1, or 100:1.

The semi-solid composition can contain surprisingly high concentration of caffeine without the taste of significant bitterness from caffeine. Caffeine content may be a total amount of caffeine and the caffeine content from the plant powder or extract. In one embodiment, the semi-solid composition may contain from about 0.5% to about 10% of caffeine. In one embodiment, the semi-solid composition comprises at least 1%, 1.5%, 2%, 2.5%, 3%, 3.5% or 4% caffeine by weight. The weight percentage of caffeine may be any number in between the ranges.

In one embodiment, the semi-solid composition comprises at least 0.5% caffeine by weight. In one embodiment, the semi-solid composition comprises at least 1% caffeine by weight. In one embodiment, the semi-solid composition comprises at least 1.5% caffeine by weight. In one embodiment, the semi-solid composition comprises at least 2% caffeine by weight. In one embodiment, the semi-solid composition comprises at least 2.5% caffeine by weight. In one embodiment, the semi-solid composition comprises at least 3% caffeine by weight. In one embodiment, the semi-solid composition comprises at least 3.5% caffeine by weight. In one embodiment, the semi-solid composition contains about 1.3% caffeine by weight. In one embodiment, the semi-solid composition contains about 0.67% caffeine by weight. In one embodiment, the semi-solid composition contains about 1.1% caffeine by weight.

The gelling component serves to form the polymeric matric providing the semi-solid texture to the semi-solid composition. The semi-solid composition may contain at least 0.5% by weight of the gelling component. In one embodiment, the semi-solid composition contains from about 1.25% by weight to about 8.5% by weight of the gelling component. In one embodiment, the semi-solid component comprises about 0.5%, 0.75%, 1.25%, 1.75%, 2.25%, 2.75%, 3.25%, 3.75%, 4.25%, 4.75%, 5.25%, 5.75%, 6.25%, 6.75%, 7.25%, 7.75%, 8.25%, or 8.75% by weight of the gelling component.

In one embodiment, the gelling component comprises gelatin, donkey hide gelatin, starch, pectin, gellan gum, gum Arabic, carrageenans, guar, agar, alginate, locust bean gum, xanthan, or derivatives thereof. In one embodiment, the gelling composition comprises pectin and gelatin in a ratio from about 10:1 to about 1:1. In one embodiment, the gelling composition comprises gelatin and starch in a ratio from about 100:1 to about 1:100. In one embodiment, the gelling composition comprises gelatin and alginate. In one embodiment, the gelling composition comprises alginate and starch. In one embodiment, the gelling composition consists essentially of starch, gelatin, alginate or pectin.

In one embodiment, the gelling component consists essentially of pectin. In one embodiment, the gelling component comprises apple pectin, citrus pectin, or a combination thereof. In one embodiment, the semi-solid composition comprises at least 1% of pectin. In one embodiment, the semi-solid composition comprises from about 1% to about 5% of pectin. In one embodiment, the semi-solid composition comprises about 2.5% pectin. In one embodiment, pectin has a methoxy content of not less than 30%, 40% or 50%. In one embodiment, pectin has an amid content of not less than 10%, 15%, 20%, 25%, 30% or 40%. In one embodiment, pectin has a carboxylic content of not less than 25%, 30%, 35%, 40%, 50%, or 60%. In one embodiment, the pectin has a methyl ester not more than 30%, 32%, 35%, or 40%.

In one embodiment, the gelling component consists essentially of gelatin or collagen. In one embodiment, the gelling component consists essentially of starch. In one embodiment, the gelling component consists essentially of alginate.

The semi-solid composition may further comprise comprising a modulating composition, an antioxidant composition, a vitamin composition, a mineral composition, an amino acid composition, an herb composition, or a combination thereof.

The modulating composition may be configured to enhance caffeine stimulant effect, reduce the jittery or cardiovascular side effects, or a combination thereof. In one embodiment, the modulating composition is configured to reduce the side effects of caffeine such as jittery and anxiety. In one embodiment, the modulating composition comprises magnesium, L-theanine, theobromine, piraletam, citicoline, flavonoids, quinones, blubbery extract or isolates, arginine, vitamin E, bacopa, curcumin, ginseng, citrulline, icariin, forsklin, S-denosyl-L-methionine, quercetine, taurine, or isolates, extracts or derivatives thereof.

In one embodiment, the modulating composition comprises gingko biloba, cocoa flavonoids, theobromine, theanine, piraletam, citicoline, blubbery extract or isolates, arginine, vitamin E, bacopa, curcumin, ginseng, citrulline, icariin, forsklin, S-denosyl-L-methionine, quercetine, taurine, or isolates, extracts or derivatives thereof. In one embodiment, the modulating composition comprises cat's claw (Uncaria tomentosa, Uncaria guianensis), lavender, cinnamon (Cinnamomum verum), Yarrow (Achilea millefolium), hawthorn, garlic, Buchu (Agathosma betulina), prickly custard apple (Annona muricata), Cassia occidentalis (Coffee weed), Hibiscus sabdariffa (Roselle), or a combination thereof. In one embodiment, the modulating composition may include ginko biloba, turmeric, ginger, astragalus, Prunella vulgaris, Pueraria montana var. lobata, Salvia miltiorrhiza, Coptis chinensis, Eucommia ulmoides Oliver, cranberry, blackberry, elderberry extract, cranberry, blueberry, grapeseed, saffron, Sangre de grado (dragon's blood), its extract, powder or derivative thereof.

In one embodiment, the modulating composition comprises gingko biloba. In one embodiment, the weight ratio of caffeine and gingko biloba is from about 1:100 to about 100:1. In one embodiment, the weight of caffeine and gingko biloba ratio is about 10:1. In one embodiment, the weight ratio of caffeine and gingko biloba is about 20:1. In one embodiment, the weight ratio of caffeine and gingko biloba is about 2:1. In one embodiment, the weight ratio of caffeine and gingko biloba is about 1:1.

In one embodiment, the antioxidant composition comprises wherein the antioxidant composition comprises vitamin A, vitamin E, vitamin C, beta-carotene, alpha-carotene, lycopene, lutein, folic acid, gallic acid, resveratrol, quinone, Coenzyme Q10, selenium, selenium yeast, phenolics, polyphenols, anthocyanins, flavonoids, astaxanthin, canthaxanthin, cryptoxanthin, anthracenes, carotenoids, zeaxanthin, curcumin, or derivatives thereof.

In one embodiment, the vitamin composition comprises vitamin A, B, C, D, E, K or a combination thereof. In one embodiment, vitamin B comprises thiamin (B1), riboflavin (B2), niacin or niacinamid (B3), pantothenic acid (B5), pyridoxines (B6), biotin (B7), folate or folic acid (B9), cobalmin (B12), or their derivative thereof.

In one embodiment, the mineral composition comprises salts of calcium, iron, zinc, magnesium, sodium, chloride, potassium, copper, molybdenum, manganese, phosphorus, iodine, nickel, or selenium, or a combination thereof.

In one embodiment, the amino acid composition comprises an essential amino acid or its derivative thereof. In one embodiment, the amino acid composition comprises branch-chain amino acids. In one embodiment, the amino acid composition comprises leucine, isoleucine, valine, their derivative or a combination thereof. In one embodiment, the amino acid composition comprises a stimulant amino acid or its derivative. Example simulant amino acids include tryptophan, aspartate, N-methyl-D-aspartate (NMDA), L-carnitine, L-theanine, glutamate, glutamine, or their derivatives thereof.

The herbal composition may add additional bioactive benefit to the gummy composition, may act synergistically with caffeine to, for example, enhance the mental stimulating effect of the gummy composition, or both. In one embodiment, the herbal composition comprises gotu kola, ginseng, St. John's wort, khat, ephedra, yerba mate (Ilex paraguariensis), guarana (Paullinia cupana), cola nut (Cola nitidalacuminata), cocoa (Theobroma cacao), gynostemma, cayenne, cloves, dandelion root, nutmeg, ashwagandha, mint, astragalus, coriander seed, maca, anise seed, tulsi, cinnamon, long pepper, hyperzine-A, rhodiola, lion's mane, oat straw, bacopa, artichoke, kratom, its extract, powder, or derivative thereof.

In one embodiment, the semi-solid composition may further include an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the sweetener comprises sucrose, fructose, glucose, erythritol, xylitol, sugar, glucose syrup, corn syrup, high fructose corn syrup, trulinose, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenyl alanine 1-methyl ester, salts thereof, licorice or its extracts or isolates, or a mixture thereof.

In one embodiment, the flavoring agent comprises vanilla, chili oil, gingerol, peperine, capsaicin, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, rosemary oil, lavender oil, sage oil, rose extra or oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, cocoa, pineapple flavor, berry flavors or mixtures or derivatives thereof. In one embodiment, the berry flavor comprises flavors, isolates, extracts, or juices of blueberry, raspberry, strawberry, black current, acai berry, bilberry, blackberry, mulberry, boysenberry, cranberry, elderberry, goji berry, gooseberry, huckleberry, or a combination thereof.

The coloring agent may be synthetic or natural. Example natural coloring agents include, without limitation, plant or fruit extract or juice or powder such as, without limitation, beet extract, strawberry extract, carrot extract, spirulina, cochineal, flower extracts or powders such as rose extract or powder, turmeric extract or powder, curcumin extract or powder.

The semi-solid composition may have a pH from slightly basic, neutral or acidic. In one embodiment, the pH of the composition is less than 3. In one embodiment, the pH of the composition is from about 2.7 to about 2.9. In one embodiment, the pH of the composition is form about 2.91 to about 2.99. In one embodiment, the pH of the composition is from about 3.0 to about 3.5. In one embodiment, the pH of the composition is from about 3.51 to about 3.99. In one embodiment, the pH of the composition is from about 4.0 to about 4.5. In one embodiment, the pH of the composition is from about 4.51 to about 4.99. In one embodiment, the pH of the composition is more than 5.0. In one embodiment, the pH of the composition is about 3 to about 5. In one embodiment, the pH of the composition is about 5 to about 7. In one embodiment, the pH of the composition is about 6 to about 8.

In one embodiment, the semi-solid composition comprises at least 1.3% by weight of caffeine. In one embodiment, the composition further comprises Ginkgo biloba, cocoa powder, and vitamin Bs. The gelling component consists comprises pectin, carrageenan, gelatin, starch, locust bean gum, xanthan gum, gum Arabic, or some combination of the above.

In one embodiment, the semi-solid composition may be raspberry, orange, coconut, pineapple, cinnamon, chili pepper, jalapeno pepper, all spice, anise, licorice, rose, peppermint, mint, caramel, salted caramel, pumpkin spice, cinnamon, or gingerbread flavored. In one embodiment, the semi-solid composition is flavored by ground or powdered spice or fruit material. In one embodiment, the semi-solid composition comprises flavors such as liquid flavorings or extracts.

In another aspect, the application provides methods of making the semi-solid caffeinated compositions disclosed therein. In one embodiment, the method including combining the caffeinated component with the complex component to provide a first mixture and combining a gelling component with sugar component and optionally buffer salts to provide a second mixture. Mixing the first mixture and the second mixture and optionally other ingredient to provide a combined mixture. Heat the combined mixture to at least brix 78. In one embodiment, heat the combined mixture to a brix from about 78 to about 83, rom about 79 to about 85, from about 80 to about 87, from about 81 to about 90. In one embodiment, heat the combined mixture to a brix less than about 90. Mold the mixture and allow to cool.

### EXAMPLES

### Example 1: Caffeinated Orange Gummy

Ingredients: Water, Glycerol, 13.6 g Caffeine, Pectin, Fructose, Sodium Citrate, Sucrose 36.5 g Bionap Red Orange Complex H, 24 g gamma-cyclodextrin, 1.5 g Ginkgo, Coconut Oil Glucose Syrup, 2.2 g B-Vitamin Premix, Citric Acid in 50% glycerol/water, and Orange Flavor

Pectin, fructose, and sodium citrate were added to a container and mixed until homogenous to provide a mixture 1. Sucrose, red orange complex H, and Ginkgo biloba were added to a separate container and mixed until homogenous to provide a mixture 2.

A mixture of water and glycerol mixture was heated to 200° F. and caffeine was and allowed to dissolve, then add the mixture 1. The solution was brought to boil. Mixture 2 was then added to provide a mixture 3.

The glucose syrup and coconut oil were mixed and brought to a boil until Brix 85-90 was reached. Then the mixture 3 was added to provide a gummy batter. The gummy batter was poured into silicone molds and allowed to cool. The gummy pieces containing about 103 mg caffeine per 7.5 gram piece.

### Example 2. Caffeinated Strawberry Orange Gummy

Ingredients: 285 g Water, 5 g Glycerol, 13.6 g Caffeine, 45 g Pectin, 100 g Fructose, 3 g Sodium Citrate, 220 g Sucrose, 36.3 g Strawberry Powder, high Nucleic Acid Content, 24.1 g gamma-cyclodextrin, 1.6 g Ginkgo, 6 g Coconut Oil, 410 g Glucose Syrup, 2.2 g B-Vitamin Premix, 25 g 50% Citric Acid in 50% glycerol/water, Mint Flavor

Pectin, fructose, and sodium carbonate were added to a container and mixed until homogenous to provide a mixture 1. Sucrose, strawberry powder, and ginkgo extract were added to a separate container and mixed until homogenous to provide a mixture 2.

Water was mixed with glycerol and heated to 200° F. and then caffeine was added and allowed to dissolve. The mixture 1 was added to the heated mixture. The total mixture was brought to a boil until all the pectin was swelled. The mixture 2 was then slowly added to the boiling mixture.

To a separate container was added Coconut oil followed by glucose syrup. The glucose syrup was heated until Brix 85-90 was reached. At which point, the boiling glucose syrup was slowly added to the boiling mixture with stirring.

The gummy batter is then poured into silicone molds and allowed to cool to room temperature at which time the gummy can be removed provide g ˜106 mg caffeine per 7.5-gram piece.

### Example 3. Caffeinated Cacao Gummy

Ingredients: Water, Glycerol, 13.6 g Caffeine, Pectin, Fructose, Sodium Citrate, Sucrose, Hershey's Cacao Powder (100%), gamma-cyclodextrin, Ginkgo, Coconut Oil, Glucose Syrup, B-Vitamin Premix, Citric Acid, and Rose Flavor

Pectin, fructose, and sodium carbonate were added to a container and mixed until homogenous to provide a mixture 1. Sucrose, 100% pure cacao powder, and ginkgo extract were added to a separate container and mixed until homogenous to provide a mixture 2.

Water and glycerol were mixed and heated to 200° F. and caffeine was added and allowed to dissolve. Then, the mixture 1 was to the heated water/caffeine mixture. The combined solution was boiled until all the pectin is swelled. The mixture 2 was then added to provide a combined mixture.

To a separate container was added Coconut oil and glucose syrup. The glucose mixture was heated until Brix 85-90. At which point, the glucose mixture is slowly added to the combined mixture to provide the gummy batter.

The gummy batter is then poured into silicone molds and allowed to cool to room temperature at which time the gummy can be removed provide ˜102 mg caffeine per 7.5-gram gummy and ˜7 mg theobromine per 7.5-gram piece.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A semi-solid composition, comprising,
a gelling component in a sufficient amount to provide a cohesive gelled product,
a caffeinated component comprising caffeine, wherein the caffeinated component is bitter, and
a complexing component, wherein the complexing component is configured to reduce the bitterness of the caffeinated component by complexing with the caffeinated component.

2. The semi-solid composition of claim 1, wherein the caffeinated component comprises caffeine, green coffee bean powder or extract, green tea powder or extract, white tea powder or extract, black tea powder or extract, guarana powder or extract, yerba mate powder or extract, cola nut powder or extract, cacao powder or extract, coffee powder or extract, or a combination thereof.

3. The semi-solid composition of claim 1, wherein the caffeinated component comprises a plant extract or powder containing at least 10% of caffeine.

4. The semi-solid composition of claim 1, wherein the caffeinated component comprises cacao derivatives, comprising theobromine, polyphenol, flavonoids, or a combination thereof.

5. The semi-solid composition of claim 1, wherein the complexing component comprises nucleic acid, nucleotide, nucleic acid base, fruit power, protein, peptide, cluster dextrin, cyclodextrin, polydextrose, polyethylene glycol, fatty acids, waxes, zeolite, chitosan, poly N-acetyglucosamine, N-acetylglucosamine, or a combination thereof.

6. The semi-solid composition of claim 1, wherein the complexing component is configured to complex with caffeine.

7. The semi-solid composition of claim 4, wherein the complexing component is configured to complex with theobromine, polyphenol, flavonoids, or a combination thereof.

8. The semi-solid composition of claim 1, wherein the semi-solid composition comprises at least 0.5% the complexing component by weight.

9. The semi-solid composition of claim 1, wherein the semi-solid composition comprises at least 3% the complexing component by weight.

10. The semi-solid composition of claim 1, wherein the complexing component comprises cyclodextrin and wherein the semi-solid composition comprises at least 0.1% of cyclodextrin.

11. The semi-solid composition of claim 1, wherein the complexing component comprises strawberry powder and wherein the semi-solid composition comprises at least 0.5% of strawberry powder.

12. The semi-solid composition of claim 1, wherein the gelling composition comprises gelatin, starch, pectin, gellan gum, gum Arabic, carrageenans, guar, agar, alginate, locust bean gum, xanthan, or a combination or derivatives thereof.

13. The semi-solid composition of claim 1, wherein the gelling composition consists essentially of pectin having a methoxy content of not less than 40%.

14. The semi-solid composition of claim 1, wherein the gelling composition consists essentially of pectin having an amid content of not less than 12%.

15. The semi-solid gummy composition of claim 1, further comprising a sugar composition, wherein the sugar composition comprises sucrose, glucose, fructose, palatinose, trehalose, psicose, tagatose, sorbose, or a combination thereof.

16. The semi-solid gummy composition of claim 15, wherein the sugar composition comprises essentially sucrose, glucose, fructose, or a combination thereof.

17. The semi-solid gummy composition of claim 15, wherein the sugar composition comprises essentially trehalose, palatinose, psicose, tagatose, sorbose, or a combination thereof.

18. The semi-solid composition of claim 1, wherein the semi-solid composition comprises at least 0.5% caffeine by weight.

19. The semi-solid composition of claim 1, wherein the semi-solid composition comprises at least 1% caffeine by weight.

20. The semi-solid composition of claim 1, further comprising a modulating composition, an antioxidant composition, a vitamin composition, a mineral composition, an amino acid composition, an herb composition, a prebiotic composition, a probiotic composition, or a combination thereof.

21. The semi-solid composition of claim 20, wherein the modulating composition comprises magnesium, L-theanine, theothromine, piraletam, citicoline, flavonoids, quinones, blubbery extract or isolates, arginine, vitamin E, bacopa, curcumin, ginseng, citrulline, icariin, forsklin, S-denosyl-L-methionine, quercetine, taurine, gingko biloba, cocoa, dangui, danshen, safflower, grape seed, turmeric, theobromine, theanine, piraletam, citicoline, blubbery extract or isolates, arginine, vitamin E, bacopa, curcumin, ginseng, citrulline, icariin, forsklin, S-denosyl-L-methionine, quercetine, taurine, cat's claw (Uncaria tomentosa, Uncaria guianensis), lavender, cinnamon (Cinnamomum verum), Yarrow (Achilea millefolium), hawthorn, garlic, Buchu (Agathosma betulina), prickly custard apple (Annona muricata), Cassia occidentalis (Coffee weed), Hibiscus sabdariffa (Roselle), or plant parts, extract, isolates or powders thereof.

22. The edible composition of claim 1, wherein the modulating composition comprises epicatechin, catechin, quercetin, kaempferol, isorhamnetin, amentoflavone, bilobetin, isoginkgetin, ginkgetin, sciadopitysin, or a combination thereof.

23. The semi-solid composition of claim 20, wherein the antioxidant composition comprises vitamin A, vitamin E, vitamin C, beta-carotene, alpha-carotene, lycopene, lutein, folic acid, gallic acid, resveratrol, quinone, Coenzyme Q10, selenium, selenium yeast, phenolics, polyphenols, anthocyanins, flavonoids, astaxanthin, canthaxanthin, cryptoxanthin, anthracenes, carotenoids, zeaxanthin, curcumin, or derivatives thereof.

24. The semi-solid composition of claim 20, wherein the vitamin composition comprises vitamin A, B, C, D, E, K or a combination thereof.

25. The semi-solid composition of claim 20, wherein the mineral composition comprises salts of calcium, iron, zinc, magnesium, sodium, chloride, potassium, copper, molybdenum, manganese, phosphorus, iodine, nickel, or selenium, or a combination thereof.

26. The semi-solid composition of claim 20, wherein the amino acid composition comprises an essential amino acid, a branch-chain amino acid, a stimulant amino acid, or its derivative thereof.

27. The semi-solid composition of claim 20, wherein the herbal composition comprises ginko biloba, turmeric, ginger, astragalus, Prunella vulgaris, Pueraria montana var. lobata, Salvia miltiorrhiza, Coptis chinensis, Eucommia ulmoides Oliver, cranberry, blackberry, elderberry extract, cranberry, blueberry, grapeseed, saffron, Sangre de grado (dragon's blood), its extract, powder or derivative thereof.

28. The semi-solid composition of claim 1, further comprising an additive selected from sweeteners, food acids, flavoring agents, coloring agents, humectants, bulking agents, fatty acids, triglycerides, plasticizers, emulsifiers, thickeners, preservatives, or and a mixture thereof.

29. The semi-solid composition of claim 27, wherein the sweetener comprises erythritol, xylitol, sucrose, fructose, glucose, maltose, juice or juice concentrate, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

30. The semi-solid gummy composition of claim 1, having a glycemic index of not more than 25.
